(19) United States
(12) Patent Application Publication
(10) Pub. No.: US 2009/0164141 A1
(43) Pub. Date: Jun. 25, 2009

(54) MULTIFUNCTIONAL PORTABLE ELECTRONIC DEVICE

(75) Inventor: TUNG-LUNG LEE, Tu-Cheng (TW)

Correspondence Address: PCE INDUSTRY, INC.;ATT. Steven Reiss, 458 E. LAMBERT ROAD, FULLERTON, CA, 92835, US

(73) Assignee: CHI MEI COMMUNICATION SYSTEMS, INC., Tu-Cheng City (TW)

(21) Appl. No.: 12/164,237

(22) Filed: Jun. 30, 2008

(30) Foreign Application Priority Data
Dec. 21, 2007 (CN) 200710203328.3

(51) Int. Cl. G01N 33/497; G06F 19/00

(52) U.S. Cl. 702/30; 73/23.3

(57) ABSTRACT

A multifunctional portable electronic device (100) that easily allows a user to test their breath-alcohol level is described. A power module (10) supplies a working voltage to an alcohol-testing module (12) and an output voltage generated according to a test result from the alcohol-testing module. A signal processor converts the output voltage of the alcohol-testing module into a digital signal and calculates out a corresponding alcohol concentration value that can be displayed on a display module (18).

### CROSS-REFERENCE TO RELATED APPLICATION

This application is related to a co-pending U.S. Patent Application (Attorney Docket No. [US17686]), entitled “MULTIFUNCTIONAL PORTABLE ELECTRONIC DEVICE”, by Tung-Lung Lee. Such application has the same assignee as the present application and is concurrently filed herewith. The disclosure of the above-identified application is incorporated herein by reference.

### BACKGROUND OF THE INVENTION

1. Field of the Invention

The present invention generally relates to portable electronic devices, and more particularly to a multifunctional electronic device.

2. Description of Related Art

Motor vehicle crashes caused by alcohol-impaired drivers are a significant social and economic problem. A driver who has been drinking may hardly notice that his/her driving ability has been impaired by the alcohol at the time, but the slow reaction time that can result from even a slight case of alcohol impairment can increase the chances of having an accident, and thus could prove fatal in an emergency driving situation.

If a driver could check the alcohol concentration in his/her body prior to operating a motor vehicle instead of simply relying on how he/she feels to determine whether his/her driving ability has been impaired by the alcohol, he/she might have second thoughts about driving after drinking, thereby reducing the chances of having an accident.

There are several ways to determine the alcohol concentration in a person's body. The alcohol concentration can be roughly estimated using a mathematical approach. One of the most effective ways of doing this is to perform a blood or urine test. However, in some situations, it is inconvenient to perform the blood or urine test. Inexpensive alcohol concentration measuring devices such as a Breathalyzer are available as a simpler and less invasive alternative to the blood or urine test.

However, a Breathalyzer is not considered a device that is widely used and carried in people's daily life. Portable electronic device such as mobile phone on the other hand, are considered a widely used electronic device that is often carried by users at all times.

In addition, a user who wants to use both a Breathalyzer and a mobile phone must carry both of them with him at all times, which is inconvenient for the user.

Therefore, a new multifunctional portable electronic device having a function of alcohol monitoring function is desirable to overcome the above-described problems.

### BRIEF DESCRIPTION OF THE DRAWINGS

Many aspects of the multifunctional portable electronic device can be better understood with reference to the following drawings. Drawings are not necessarily drawn to scale, the emphasis instead being placed upon clearly illustrating the principles of the present multifunctional portable electronic device. Moreover, in the drawings, like reference numerals designate corresponding parts throughout the several views, in which:

FIG. 1 is a block diagram of a multifunctional portable electronic device according to an examplary embodiment of the invention;

FIG. 2 is a circuit diagram of a alcohol-testing module of the multifunctional portable electronic device shown in FIG. 1; and

FIG. 3 a isometric view of a multifunctional portable electronic device shown in FIG. 1.

### DETAILED DESCRIPTION OF THE EMBODIMENTS

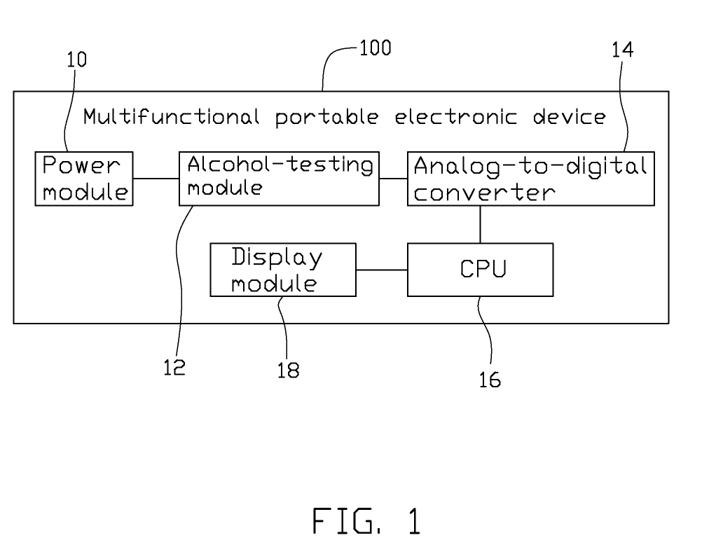

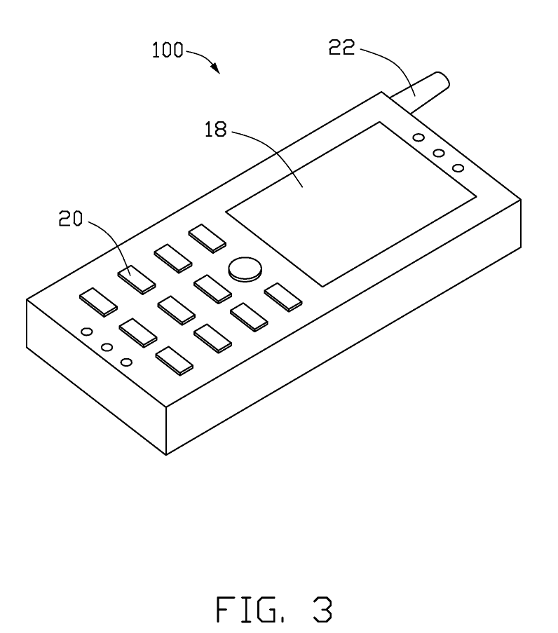

Referring to FIGS. 1 and 3, an exemplary embodiment of a multifunctional portable electronic device 100 in the form of a mobile phone is shown. The multifunctional portable electronic device 100 includes a power module 10, an alcohol-testing module 12, an analog-to-digital converter 14, a center processing unit (CPU) 16, a display module 18, a keypad 20 and an antenna 22. The power module 10, the alcohol-testing module 12, the analog-to-digital converter 14, the CPU 16 and the display module 18 are electronically connected in series. The power module 10 supplies power for operating the multifunctional portable electronic device 100. The keypad 20 allows inputting information. The antenna 22 receives and transmits signals.

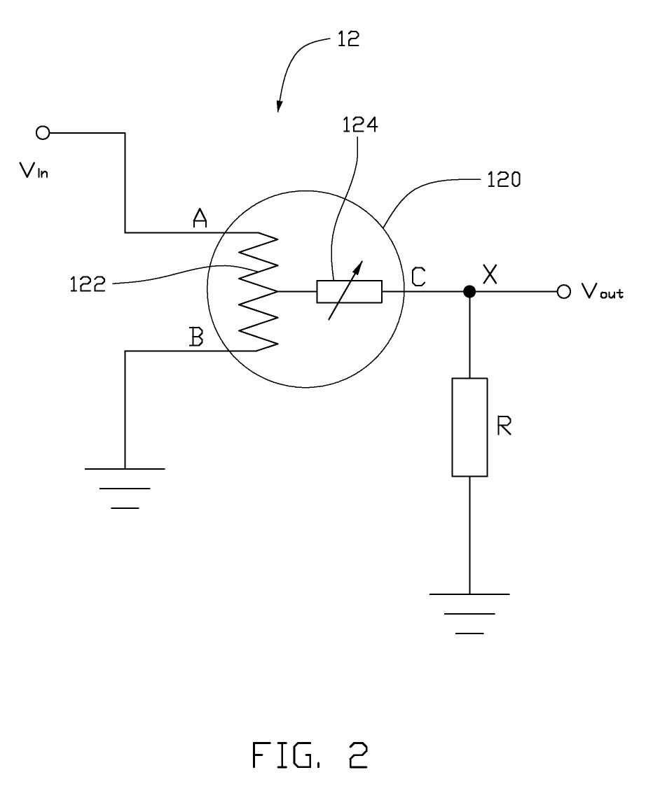

Referring to FIG. 2, the alcohol-testing module 12 includes an alcohol-sensing portion 120 and a load resistor R. The alcohol-sensing portion 120 is a conventional semiconductor gas sensor designed for breath alcohol concentration detection. The alcohol-sensing portion 120 includes a heater 122 and a detector 124 and has a first electrical contact point A, a second electrical contact point B and a third electrical contact point C. The two ends of the heater 122 are connected to the first electrical contact point A and the second electrical contact point B, respectively. The middle portion of the heater 122 is connected with one end of the detector 124. The other end of the detector 124 is connected to the third electrical contact point C.

In the exemplary embodiment, the heater 122 is an inductance heater, and when in use, the heater 122 radiates heat. The detector 124 includes tin dioxide, a standard substance for detecting alcohol. The first electrical contact point A is an input end of the alcohol-sensing portion 120 and is connected to the working voltage Vᵢₙ of the alcohol-sensing portion 120, wherein the working voltage Vᵢₙ is supplied by the power module 10. The second electrical contact point B is connected to ground. The third electrical contact point C is connected with the load resistor R. When in use, the voltage on the load resistor R is an output voltage Vₒᵤₜ of the alcohol-testing module 12. A node X is defined between the third electrical contact point C and the load resistor R. In the exemplary embodiment, the other end of the load resistor R is connected to the ground. Therefore, the voltage of node X is equal to the output voltage Vₒᵤₜ of the alcohol-testing module 12. It should be understood that, if the load resistor R is grounded, the alcohol-sensing portion 120 works best when a voltage at the other end of the load resistor R is in a range of about 0˜5V.

The resistance Rs of the detector 124 can changed by a variance of alcohol concentration value. The detector 124 can have different resistances Rs corresponding to different sensed alcohol concentration. Meanwhile, the changing resistance Rs of the detector 124 results in changes of the output voltage Vₒᵤₜ.

Referring to FIGS. 1 and 2, the power module 10 is electronically connected with the first electrical contact point A of the alcohol-sensing portion 120, for supplying a working voltage Vᵢₙ to the alcohol-sensing portion 120. The working voltage Vᵢₙ is 0.9V±0.05V. When the working voltage Vᵢₙ is supplied to the alcohol-testing module 12, the heater 122 radiates heat allowing alcohol to be easily absorbed by the detector 122. When the detector 122 detects alcohol from the user's breath, the resistance Rₛ of the detector 122 changes in response to the alcohol concentration of the user's breath. The output voltage Vₒᵤₜ value of the alcohol-testing module 12, being an analog signal, varies with value of the resistance Rₛ. The analog-to-digital converter 14 converts the output voltage Vₒᵤₜ into a digital signal usable by the CPU 16.

The CPU 16 calculates a corresponding alcohol concentration value based on the received digital signal, and sends the alcohol concentration value to the display module 18. The display module 18 has a display screen (not shown). The tested alcohol concentration value is then displayed in the display screen of the display module 18.

It should understood that, the multifunctional portable electronic device can also be a personal digital assistant (PDA), and another portable electronic devices. The analog-digital converter 14 and the CPU 16 can be replaced by a signal processor with analog-to-digital converting function and data processing function, such as a single chip, a integrate circuit, and so on.

It is to be further understood that even though numerous characteristics and advantages of the present embodiments have been set forth in the foregoing description, together with details of the structures and functions of the embodiments, the disclosure is illustrative only, and changes may be made in detail, especially in matters of shape, size, and arrangement of parts within the principles of the invention to the full extent indicated by the broad general meaning of the terms in which the appended claims are expressed.

What is claimed is:

1. A multifunctional portable electronic device comprising:
a power module;
an alcohol-testing module electronically connected to the power module, the power module supplying a working voltage to the alcohol-testing module and the alcohol-testing module generating an output voltage according to a test result from the alcohol-testing module;
a signal processor electronically connected with the alcohol-testing module, the signal processor converting the output voltage of the alcohol-testing module into a digital signal used for calculating a corresponding alcohol concentration value; and
a display module electronically connected with the alcohol-testing module for displaying the alcohol concentration value received from the signal processor.

2. The multifunctional portable electronic device of claim 1, wherein the signal processor includes an analog-to-digital converter and a CPU electronically connected to each other, the analog-to-digital converter is connected to the alcohol-testing module, and the CPU is connected to the display module.

3. The multifunctional portable electronic device of claim 2, wherein the alcohol-testing module includes an alcohol-sensing unit and a load resistor, and has a first electrical contact point connected with the power module, a second electrical contact point connected to ground and a third electrical contact point connected with one end of the load resistor, the voltage across the load resistor comprising the output voltage of the alcohol-testing module.

4. The multifunctional portable electronic device of claim 3, wherein the other end of the load resistor grounded, and a node is defined between the load resistor and the third electrical contact point, a voltage output from the node is equal to an output voltage of the alcohol-testing module.

5. The multifunctional portable electronic device of claim 3, wherein the alcohol-sensing unit includes a heater and a detector, the two ends of the heater are respectively connected to the first electrical contact point and the second electrical contact point, the middle of the heater is connected with one end of the detector, the other end of the detector is connected with the third electrical contact point.

6. The multifunctional portable electronic device of claim 3, wherein the alcohol-sensing unit includes a semiconductor gas sensor.

7. The multifunctional portable electronic device of claim 6, wherein the alcohol-sensing unit includes a heater and a detector, the two ends of the heater connected to the first electrical contact point and the second electrical contact point, respectively, the middle of the heater connected to one end of the detector, and the other end of the detector is connected to the third electrical contact point.

8. The multifunctional portable electronic device of claim 7, wherein the detector includes an alcohol detecting material, and when used, the resistance of the detector changes in response to the amount of alcohold detected.

9. The multifunctional portable electronic device of claim 7, wherein the detector includes tin dioxide as a detecting material.

10. The multifunctional portable electronic device of claim 9, wherein the heater is an inductance heater.

11. The multifunctional portable electronic device of claim 7, wherein the output voltage of the alcohol-sensing unit is an analog signal fed into the analog-to-digital converter, the analog-to-digital converter converts the output voltage into a digital signal usable by the CPU, the CPU calculating a corresponding alcohol concentration value based on the digital signal received from the analog-to-digital converter, and the CPU sends the alcohol concentration value to the display module.

12. The multifunctional portable electronic device of claim 1, wherein the working voltage of the alcohol-sensing unit is about 0.9V±0.05V.
